(19) United States
(12) Patent Application Publication
(10) Pub. No.: US 2008/0021297 A1
(43) Pub. Date: Jan. 24, 2008

(54) Method,a System for Generating a Spatial Roadmap for an Interventional Device and Quality Control System for Guarding the Spatial Accuracy Thereof

(75) Inventor: Marcel Boosten, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONIC, N.V., EINDHOVEN (NL)

(21) Appl. No.: 10/597,749

(22) PCT Filed: Feb. 3, 2005
(86) PCT No.: PCT/IB05/50451

§ 371 Date: May 2, 2007

(30) Foreign Application Priority Data
Feb. 10, 2004 (EP) 04100488.8

(51) Int. Cl. A61B 5/04

(52) U.S. Cl. 600/374; 600/509; 607/122

(57) ABSTRACT

The invention relates to a method, a system for generating a spatial roadmap for an interventional device and a quality control system for guarding the spatial accuracy thereof. In an embodiment of the system 100 for practicing the invention an X-ray imager 100a is used for acquiring suitable images Dᵢ₋₁, Dᵢ, . . . , D_{N}, showing the volume under examination, comprising the catheters 182a, 182b. These X-ray images are then processed by means of per se known reconstruction method to yield a motion-corrected three-dimensional volume of examination. This volume is then presented by means of suitable user-interface 181 on a display unit 183 together with distal portions of the catheters 182a, 182b provided with detectable markers (for simplicity only one detectable marker per catheter is shown). The motion-corrected three-dimensional image of the target organ 184 is used to construct the motion-corrected target organ-oriented three-dimensional coordinate system which is then used for drawing the spatial roadmap 183 and which is also used to locate a spatial position of a displaceable catheter 185, provided with a further detectable marker 185′. These computations are carried out using computing means 160. The computing means 160 can be further arranged to carry out a further computation comprising a computation of a spatial discrepancy between the envisaged spatial roadmap 183 and the position of the displaceable catheter 185′. In case a substantial discrepancy is signalled and in case the catheters are positioned within the target organ by means of a controllable navigation system 190, the computing means calculates a control signal S to be applied to the navigation system 190 to correct for the mismatch between the spatial roadmap 183 and the position of the displaceable catheter 185. The control unit then applies a correction signal S to the navigation system 190 after which an interventional procedure carries on.

The invention relates to a method for generating a spatial roadmap representing an envisaged trajectory of an interventional device within a target organ, said method comprising the step of providing a catheter arranged with detectable markers within the target organ.

The invention further relates to a system for generating a spatial roadmap representing an envisaged trajectory of an interventional device within a target organ, said system comprising a catheter arranged with detectable markers, said detectable markers being conceived to be positioned within the target organ, a data acquisition system arranged to acquire image data comprising the detectable markers.

The invention still further relates to a quality control system arranged to guard a spatial accuracy of a system for generating a spatial roadmap representing an envisaged trajectory of an interventional device within a target organ.

An embodiment of a method as is set forth in the opening paragraph is known from WO 94/16623. The known method is applicable in the field of cardiac electrophysiology. In the known method two reference catheters arranged with detectable markers are inserted into a target organ of interest of a patient after which the patient is irradiated with mutually intercepting scanning beams emanating from two X-ray sources. In the known embodiment the detectable markers comprise X-ray sensitive material, for example a scintillating crystal, which is arranged to provide a signal outside of the body of the patient upon absorption of X-rays in which it is disposed. The position of the catheter in three-dimensions within the target organ is obtained by establishing a spatial position of the detectable marker, which is carried out by means of a control unit which comprises a coincidence detector arranged to correlate the output signals from the detectable markers with the corresponding scan address information from scan controllers of both X-ray units. In the known method, for mapping purposes a mapping catheter is used, which spatial position is determined with respect to two reference catheters.

It is a disadvantage of the known method that the accuracy of the mapping process is highly dependent on interrelation between the smallest pixel of the scanning X-ray beam and the size of the detectable markers.

It is an object of the invention to provide a method for generating a spatial roadmap representing an envisaged trajectory of an interventional device, whereby this trajectory is obtained with high spatial accuracy and by substantially conventional imaging means.

To this end the method according to the invention comprises the steps of:
acquiring image data of detectable markers arranged within the target organ;
constructing a motion-corrected target organ-oriented three-dimensional coordinate system using said image data;
deriving a respective spatial position information of the detectable markers within the motion-corrected target organ-oriented three-dimensional coordinate system;
constructing the spatial roadmap within the target organ by interrelating the respective spatial position information of the detectable markers.

According to the method of the invention an internal, motion-corrected organ-oriented coordinate system is constructed. This technical measure is based on the insight that due to the fact that the envisaged objectives of the interventional procedure are located on the moving target the positioning accuracy is improved with respect to the systems which use a stationary world coordinate system, like in the known method. The motion-corrected target organ-oriented three-dimensional coordinate system is preferably constructed using a motion-corrected three-dimensional volume imaging method using conventional imaging techniques, described in a European patent application EP03 100646.3, assigned to the present proprietor, whereby the detectable markers are used as features on which the motion correction is based.

Additionally, using conventional imaging techniques, like wide X-ray beams or MR-acquisition, the spatial resolution of determination of the position of the detectable marker is improved, as all volume elements of a region of interest under consideration are passed by the imaging matter, in contrast to the known method, where the scanning beam of miniature diameter is applied. It must be noted that for a performance of the method according to the invention it is sufficient to acquire images on which just the detectable markers are recognizable. This can be accomplished with a very low dose X-ray exposure, as the majority of interventional catheters currently present on the market are equipped with radio-opaque markers with substantial dimensions. Optionally, the images can be acquired with a higher image quality enabling a true three-dimensional reconstruction of the target organ, thus improving three-dimensional clinical insight of the clinician during the intervention. It must be noted that the method according to the invention is applicable to a variety of interventions, not limited to cardio electrophysiology. When the motion-corrected target organ-oriented three-dimensional coordinate system is obtained, the spatial roadmap is constructed within this coordinate system using suitable supplementary information, like tissue properties or any other suitable information. Spatial position information of the detectable markers preferably comprises respective coordinates of each detectable marker within the motion-corrected target organ-oriented three-dimensional coordinate system. Alternatively, as distances between the detectable markers on the catheter are pre-determined, the spatial position information can be formed using relative distances between the markers and an absolute coordinate of one marker. By interrelating the respective spatial position information of the detectable markers a three-dimensional trajectory of the spatial roadmap is obtained. The spatial coordinates defining the trajectory of the spatial roadmap can be absolute, or can be defined as reference to the coordinates of the detectable markers.

In an embodiment of the method according to the invention the method further comprises the steps of:
acquiring a set of readings at their respective measurement locations within the target organ using an interventional measurement catheter;
presenting the set of readings on the spatial roadmap.

It is found to be particularly advantageous when the method according to the invention is carried out in a frame of a electrophysiology to present the results of cardiac potential measurements on the spatial roadmap. This feature is enabled, for example, due to an a-priori knowledge of a spatial relation between the detectable markers and the measurement points of the measurement catheter. It must be recognized that a variety of configurations is possible, including a single catheter equipped with a plurality of gauges, or a plurality of catheters with a single measuring wire. By presenting the result of the measurement of the cardiac action potentials together with the spatial roadmap an extra control of the roadmap calculation is enabled. Preferably, the measurement results are presented in colour using a suitable graphic user interface.

In a still further embodiment of the method according to the invention, the method comprises the following steps:
acquiring further image data of a displaceable catheter in the target organ for a dwell position of the displaceable catheter, said displaceable catheter comprising further detectable markers, said further image data comprising images of detectable markers and further detectable markers;
deriving further respective spatial position information of the further detectable markers of the displaceable catheter within the motion-corrected target-organ oriented three-dimensional coordinate system.

In case an ablation procedure is envisaged using a displaceable ablating catheter, it is advantageous to provide means of real-time catheter tracking. By means of acquiring further images the spatial position information, for example, coordinate of the displaceable catheter is determined, the detectable markers being used as reference points of the motion-corrected target-organ oriented three-dimensional coordinate system. Preferably, an ECG-triggered low-dose bi-plane image acquisition is carried out for this purpose. The absolute value of the exposure is selected just enough to enable a visualization of all markers in question. Optionally, the dose can be increased to enable clinical viewing of the target organ in three-dimensions. A certain dwell position of the displaceable marker can be established with high accuracy by extracting the detectable markers of all catheters within the image and by matching this information with the already created three-dimensional coordinate system.

In a still further embodiment of the method according to the invention the method further comprises the step of matching further respective spatial position information to the spatial roadmap automatically.

It is found to be of a particular advantage to provide a visual feedback of a degree of conformance of the spatial position of the displaceable catheter to the spatial roadmap. Preferably, this is carried out by suitable graphical means, like a presentation of colour-coded lines representing the spatial roadmap, respectively the spatial position of the catheter. The operator can then insure that the ablating catheter is properly inserted and can carry on the intervention. In case a substantial discrepancy between the position of the catheter and the spatial roadmap is detected, the operator can correct it in due time, thus avoiding mistakes.

In a still further embodiment of the method according to the invention for purpose of derivation of a motion-corrected target organ-oriented three-dimensional coordinate system an image acquisition by means of a rotational scan of an X-ray source around the target organ is carried out.

It is found to be advantageous to base a three-dimensional reconstruction of the spatial position of the markers based on multiple projections as it increases the accuracy of the motion-corrected coordinate system. It must be understood that a term rotational scan refers to an image acquisition mode wherein a source of X-rays is moved through space along a certain trajectory. This trajectory can be a circle, an ellipse, or even more complex movement trajectories, for example, combining concentric movements with ellipse movements. In case a magnetic resonance imaging apparatus is used, a plurality of imaging slices including all detectable markers are used for three-dimensional reconstruction.

A system for generating a spatial roadmap representing an envisaged trajectory of an interventional device within a target organ according to the invention comprises:
computation means arranged to:
   construct a motion-corrected target organ-oriented three-dimensional coordinate system based on said images;
   derive a respective spatial position information of the detectable markers within the motion-corrected target organ-oriented three-dimensional coordinate system;
   construct the spatial roadmap within the target organ by means of interrelating the respective spatial position information of the detectable markers.

The system according to the invention enables an accurate determination of a spatial position of the envisaged trajectory due to the fact that a target organ-oriented motion corrected three-dimensional coordinate system is built up using detectable markers which can be visualised on suitable images with high detection precision, said coordinate system being constructed within the target object. Suitable imaging modalities comprise X-ray, magnetic resonance, ultra-sound and other modalities suitable for imaging tissues together with objects dispersed therein. In case the spatial roadmap is arranged to represent a burning path for an ablating catheter, it is constructed based on additional data, like measurements of cardiac potentials, which may be or may not be visually represented together with the roadmap.

In an embodiment of the system according to the invention the system further comprises a displaceable catheter conceived to be displaceably arranged within the target organ, said displaceable catheter being arranged with further detectable markers, the data acquisition means being further arranged to acquire further image data of the detectable markers and the further detectable markers for a dwell position of the displaceable catheter, the computation means being further arranged to derive further respective spatial positions of the further detectable markers within the motion-corrected target organ-oriented three-dimensional coordinate system.

For purposes of electrophysiology, the ablating catheter is being displaced in a volume of a cardiac chamber, following the spatial roadmap. Therefore, it is advantageous to obtain the three-dimensional coordinates of the ablating catheter in real time, which can be achieved by using the detectable markers as reference points to assign the ablating catheter to the same motion corrected three-dimensional coordinate system. Preferably, the system according to the invention is arranged to match the thus established spatial position of the catheter to the spatial roadmap and to signal to the operator upon an event there is a mutual displacement. Still preferably, the positioning of the catheter and the displaceable catheter is controlled by means of a suitable navigation system, per se known in the art. Preferably, the navigation system is a stereotactic navigation system. In this case the computing means of the system according to the invention is preferably arranged to control the stereotactic navigation means in order to conform the spatial position of the displaceable catheter to the desired spatial roadmap. Still preferably, the system according to the invention comprises a suitable user interface, for example a suitably arranged computer program, to feed-back the procedure to the operator. Preferably, a three-dimensional image of the spatial roadmap and the spatial position of the catheter and/or the displaceable catheter are being presented. In case the data acquisition was carried out with sufficient resolution, a three-dimensional clinical image of the target organ is preferably presented as well.

A quality control system according to the invention comprises:
means for monitoring a spatial position of the detectable markers;
means for signalling a displacement of any of the detectable markers during an intervention;
means for calibration of the motion-corrected organ-oriented three-dimensional coordinate system to yield a new motion-corrected organ-oriented three-dimensional coordinate system;
means for calibration of the spatial roadmap for the new motion-corrected organ-oriented three-dimensional coordinate system.

It is found to be of a particular importance to provide a system control, wherein the accuracy of the procedure is being monitored. For this purpose the quality control system according to the invention comprises means for monitoring a spatial position of the detectable markers. It is a common practice to perform image acquisition during the course of the intervention. The means for monitoring is arranged to check the invariability of the mutual position of the markers. This invariability can be for example checked by initially fitting the markers to a certain geometrical figure and by consecutively analyzing possible transformations of this geometrical figure. In a simpler embodiment, it is possible to store a matrix of distances or vectors describing positions of the markers in three-dimensions. In case is it detected that the mutual configuration of the markers has changed, the quality control system activates the signalling means which is arranged to warn the operator or any other suitable person about a change in the internal configuration of the markers. The quality control system according to the invention further enables a correction for the displacement. For this purpose a the markers that have been moved are notified, a new coordinate system is built-up, followed by a calibration of the spatial position of the roadmap, after which the intervention can be resumed.

In an embodiment of the quality control system according to the invention said system further comprises means for conforming a path of the displaceable catheter to the spatial roadmap. This feature can comprise a calculation of a necessary displacement of the catheter, which is made available to the operator by means of a suitable user interface. Preferably, in case the displaceable catheter is being positioned by means of a navigation system, the means for conforming a path of the displaceable catheter to the spatial roadmap being arranged to communicate to said navigation system.

These and other aspects of the invention will be explained in further detail with reference to figures, whereby like numerals or characters refer to like features.

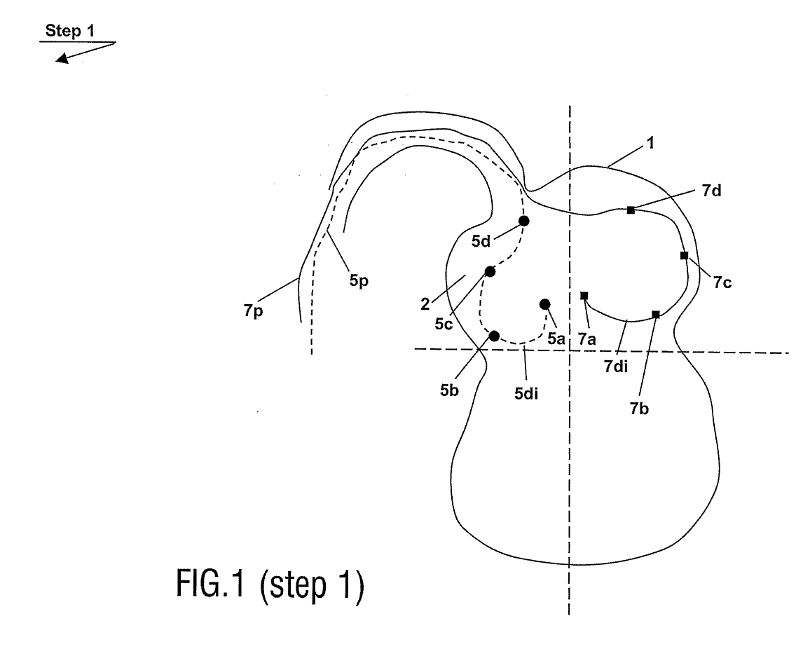

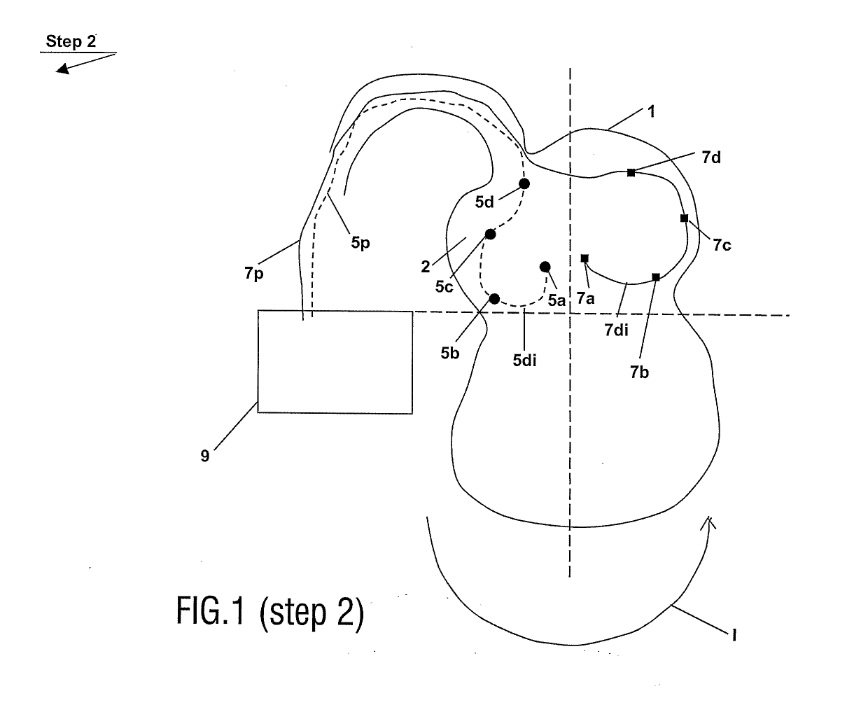

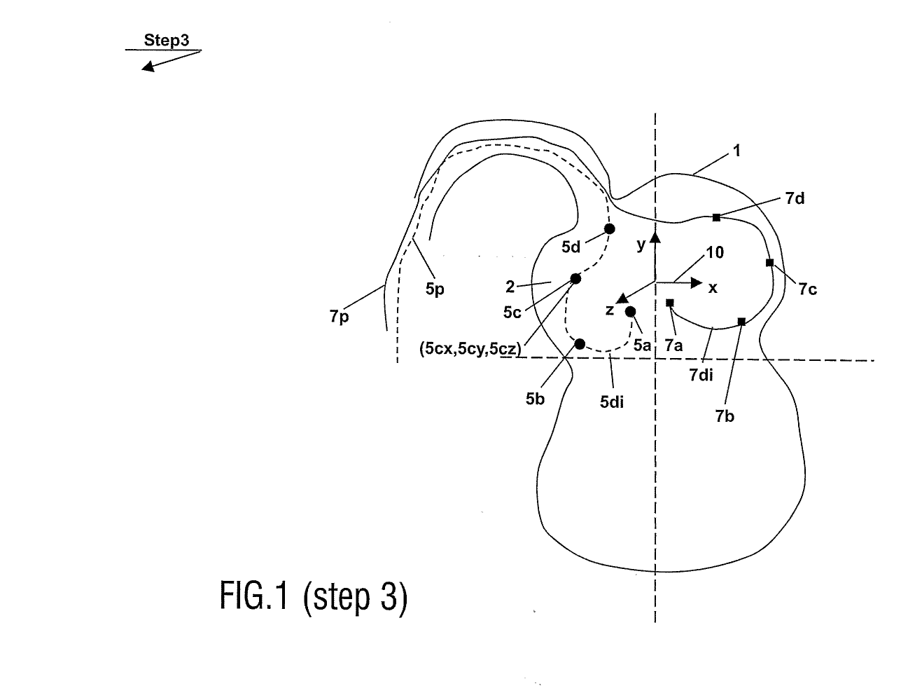

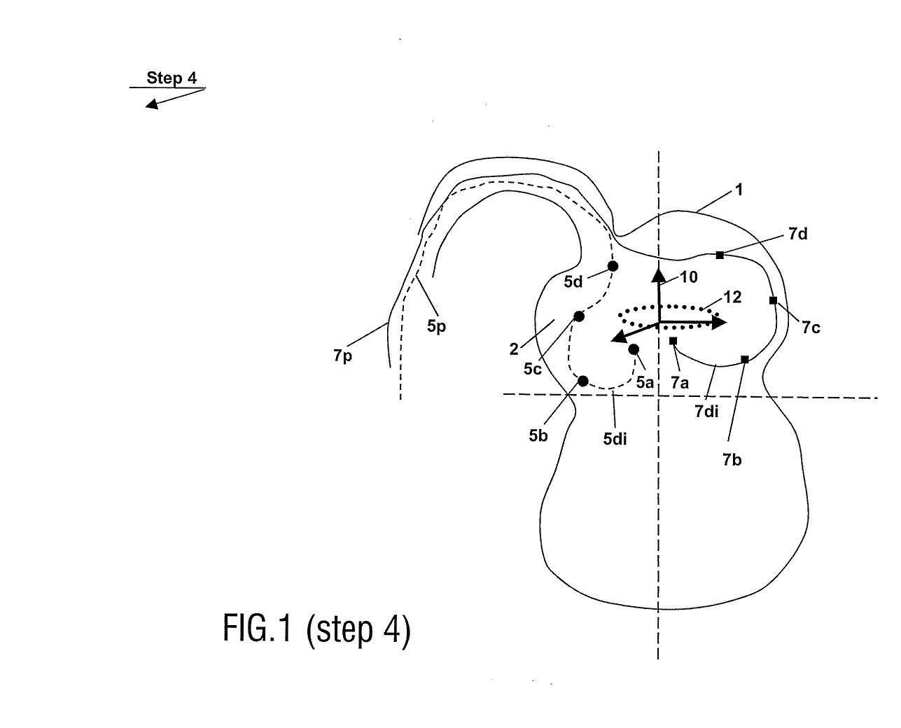

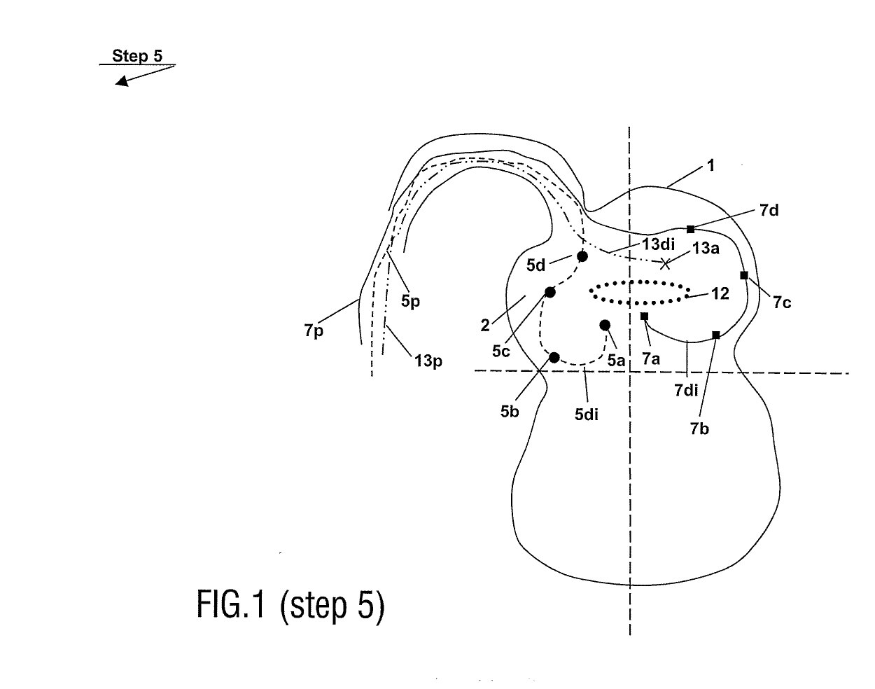

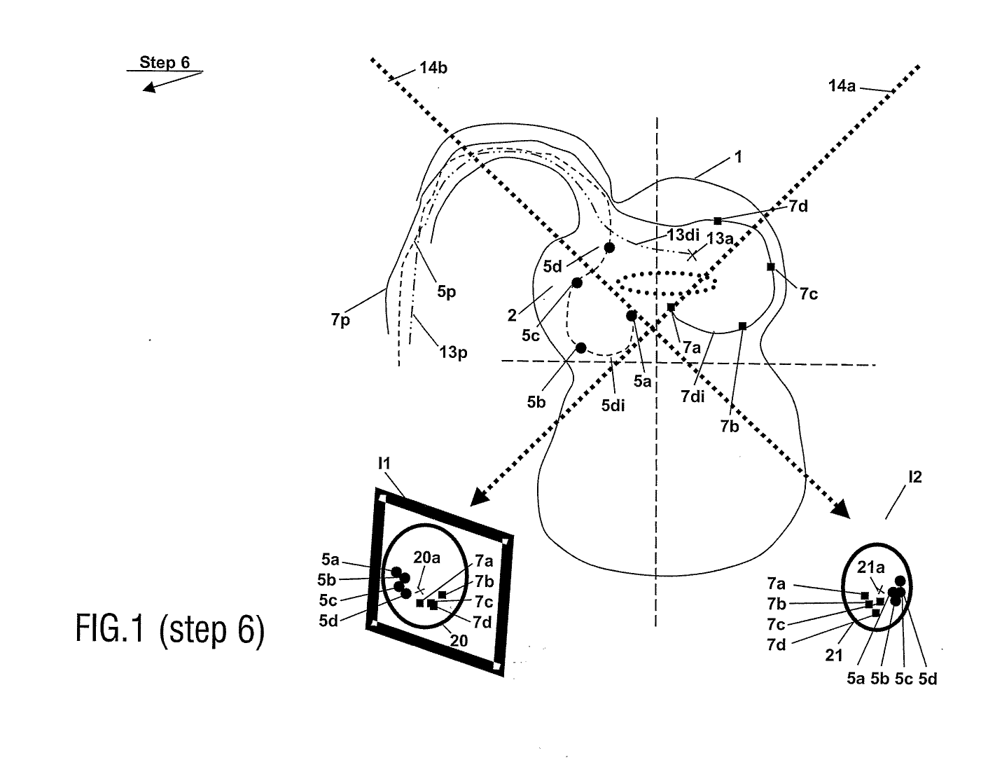

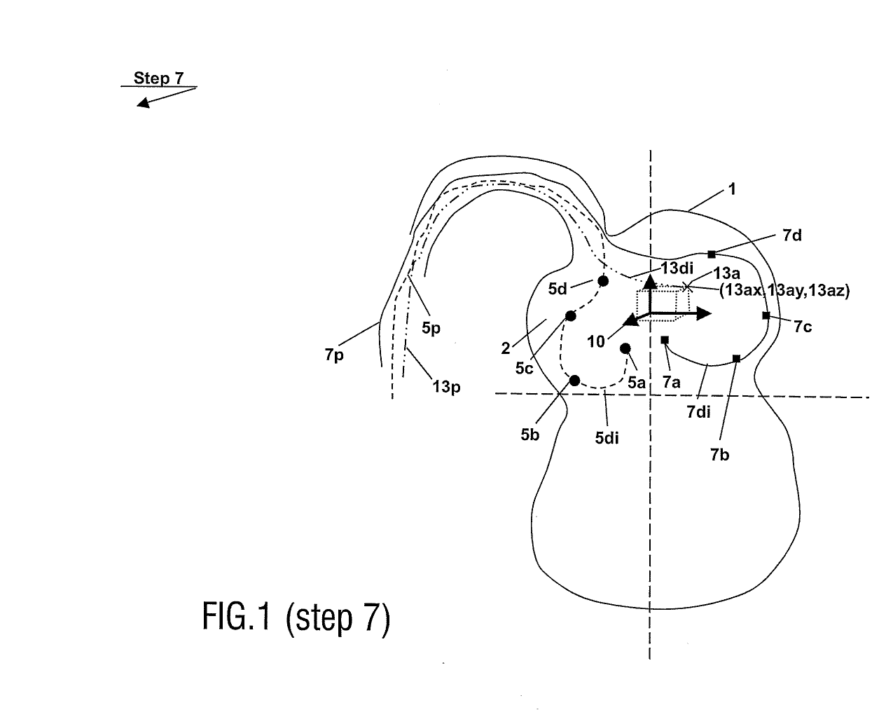

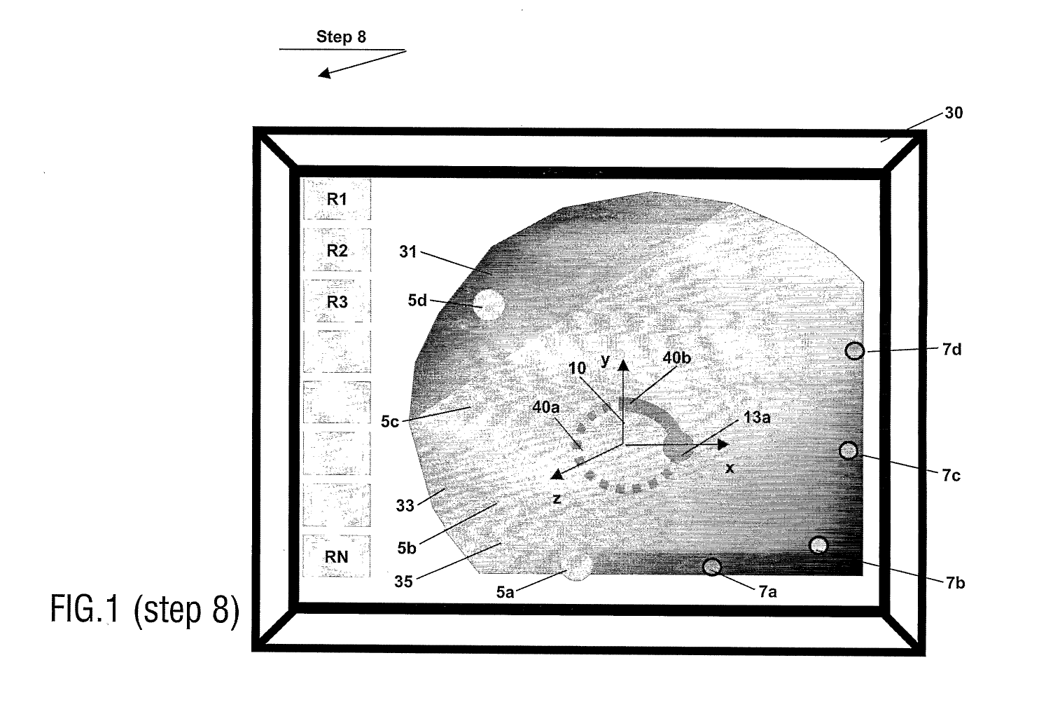

FIG. 1 presents a schematic overview of an embodiment comprising a plurality of steps of the method according to the invention.

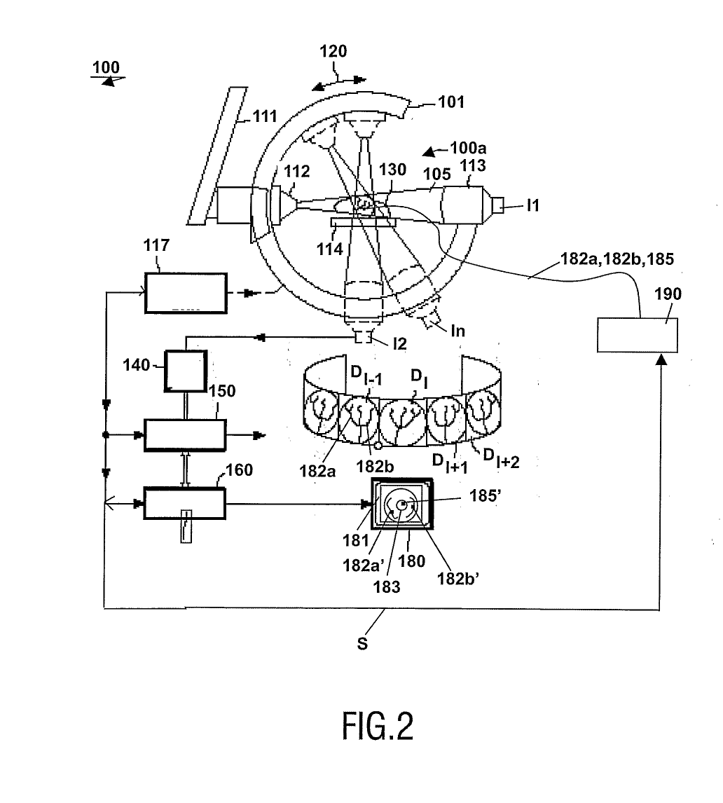

FIG. 2 presents a schematic view of an embodiment of a system according to the invention.

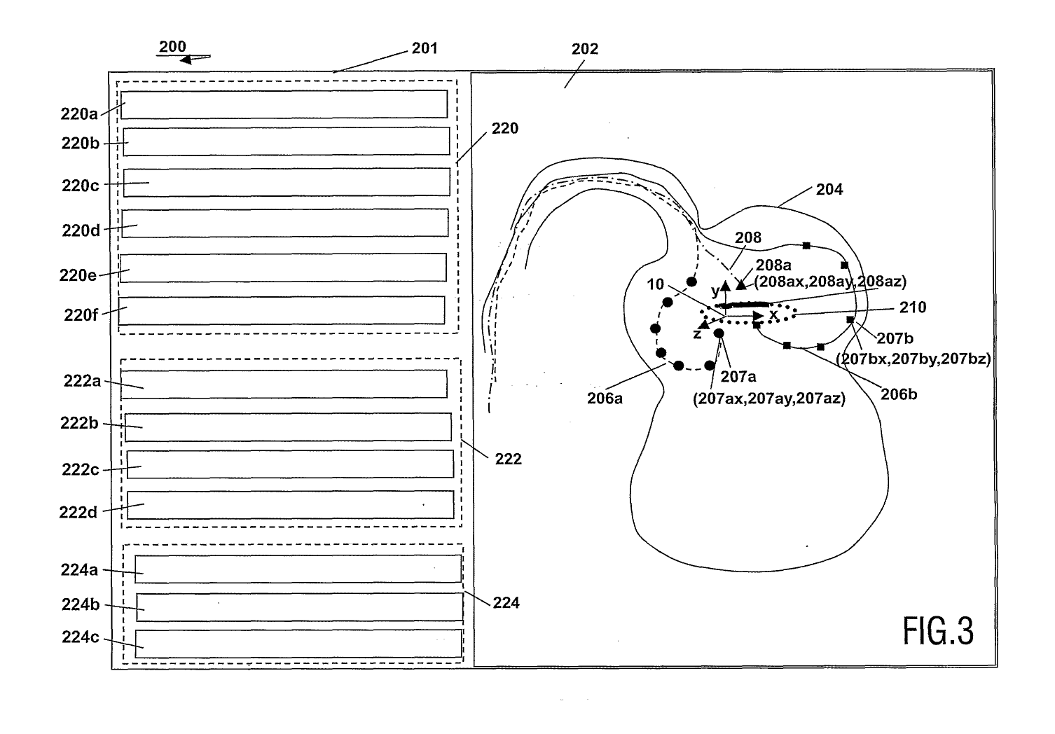

FIG. 3 presents a schematic view of an embodiment of a user interface of a system according to the invention.

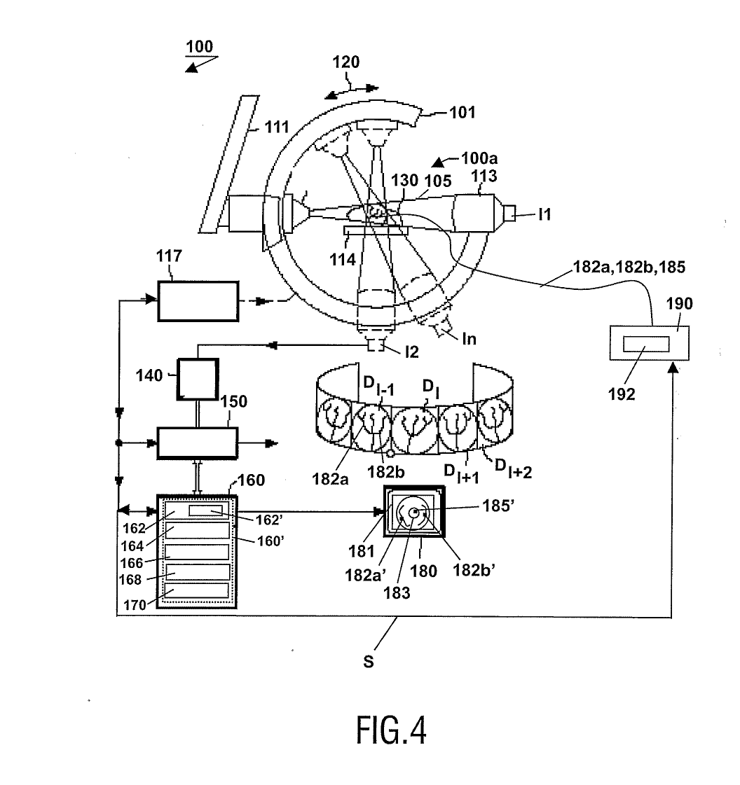

FIG. 4 presents a schematic view of an embodiment of a quality control system according to the invention.

FIG. 1 presents a schematic overview of an embodiment comprising a plurality of steps of the method according to the invention. The method according to the invention is suitable for carrying out a broad variety of interventional procedures where an accurate mapping of the organ 1 under consideration is required. For example, in the field of electrophysiology there is an objective to bum a certain geometrical figure in the flesh of a cardiac chamber. A plurality of geometrical figures is possible, including but not limited to a line, a circle, an ellipse, a square, a polygon, etc. Initially, at step 1, as a preparation for practicing the method according to the invention, a clinician inserts suitable catheters into the heart chamber 2. The catheters have a proximal portion 5p, 7p, respectively and a distal portion 5di, 7di. The distal portion of each catheter is provided with a plurality of detectable markers 5a,5b,5c,5d and 7a,7b,7c,7d in order to enable a visualization of the catheter using suitable imaging means. In spite of the fact that two catheters in the organ 1 are illustrated, it is possible to work with a larger number of catheters without departing from the teaching of the invention. Also, a number of detectable markers per catheter may vary. Preferably, the catheters are positioned in such a way that the detectable markers 5a,5b,5c,5d,7a,7b,7c,7d are substantially evenly distributed within the volume of the cardiac chamber 2 under investigation. In a conventional set-up an X-ray imaging is envisaged. In this case the detectable markers comprise radio-opaque material. Such catheters as known per se in the art. It is also possible to practice the method of the invention using magnetic-resonance imaging or ultra-sound techniques. In these cases the detectable markers are designed in accordance with corresponding principles of interaction between the imaging matter and the material of the markers. When the distal portions 5di, 7di are positioned within the cardiac chamber 2, a temporal electrical activity of the heart is measured. By relating the time moments of electrical activity of the different points of the measurement, the pattern of the contraction of the heart can be derived and possible shortcuts or irregularities in the conductivity of electrical signals can be identified. This information can be used as supplementary information for constructing the spatial roadmap.

At step 2 of the method according to the invention, image data I of at least of the cardiac chamber 2 provided with the catheters is acquired. Preferably, the catheters are held in place using suitable catheter navigation system 9. In the present illustration a rotational scan using X-ray source is depicted. However, it is sufficient to use just two orthogonal projections. In case a different imaging modality is used, for example a magnetic resonance imaging, a corresponding image acquisition is performed, said image acquisition comprising volumetric data, which is then used to carry out a 3D image reconstruction. The image reconstruction is carried out with a corresponding motion correction, whereby the detectable markers are used as features for matching. The motion correction for purposes of 3D reconstruction is described in a European patent application EP03100646.3, assigned to the same proprietor.

As a result, at step 3 a motion-corrected target organ-oriented coordinate system 10 is provided. The motion-corrected target-organ oriented coordinate system 10 has an advantage that it enables an accurate mapping of the internal surface of the moving object, like the cardiac chamber 2. The motion-corrected target organ-oriented coordinate three-dimensional system 10 is used to derive respective spatial position information of the detectable markers. Preferably, an absolute coordinate x,y,z for each detectable marker within the motion-corrected target organ-oriented coordinate three-dimensional system 10 is used as the spatial position information. For the sake of clarity of the figure the coordinate for only the marker 5c is illustrated as (5cₓ,5c_{y},5c_{z}). Naturally, each marker from the set 5a-5d, 7a-7d is assigned its coordinate within the motion-corrected target organ-oriented coordinate three-dimensional system 10.

At step 4, provided with the motion-corrected target organ-oriented coordinate system 10, the spatial roadmap 12 is constructed by interrelating the respective spatial position information of the detectable markers 5a,5b,5c,5d,7a,7b,7c,7d and by using supplementary information. Preferably, by means of a suitable graphic user interface a clinician practicing the intervention has a possibility to alter or redraw the spatial roadmap, if required. The spatial roadmap 12 is used by the clinician in a later phase of the intervention as a visual guide for steering the interventional device.

In another embodiment of the method according to the invention, the procedure explained with reference to FIG. 1 step 1—FIG. 1 step 4 comprises a plurality of additional steps.

Accordingly, at a further preparatory step 5 a displaceable catheter comprising a distal portion 13di and a proximal portion 13p is inserted into the cardiac chamber 2. Preferably, the catheters and/or the displaceable catheter are positioned within the cardiac chamber 2 by means of a suitable navigation system 9. Preferably a stereotactic navigation system is used. The distal portion of the displaceable catheter 13di comprises a further detectable marker 13a. It is also possible that the distal portion of the displaceable marker comprises a plurality of further detectable markers of the kind 13a. For purposes of electrophysiology, the function of the displaceable catheter is to burn a pattern in the flesh of the cardiac chamber according to the spatial roadmap derived during steps 1-4 of the method according to the invention.

At step 6 of the method according to the invention, a further image acquisition of the target organ comprising the distal portions of the catheters and the distal portion of the displaceable catheter is acquired. In case the image acquisition is carried out by means of X-ray imaging, it is sufficient to obtain two transmission images for orthogonal projections, as is depicted by 14a, 14b. The resulting images I1, I2 thus comprise at least all detectable markers 5a-5d, 7a-7d and the further detectable marker, 20a, 21a, respectively. Optionally, the images I1, I2 also comprise anatomical data 20, 21.

At step 7, the detectable markers and the further detectable marker are extracted from the images I1, I2 and are assigned respective spatial position information. This spatial position information is then matched to the already created motion-corrected target-organ oriented three-dimensional coordinate system 10. As a result the spatial position information (13aₓ,13a_{y},13a_{z}) of the displaceable catheter 13di is established with high precision. When the distal portion 13di of the displaceable catheter is moved, the steps 6 and 7 are repeated to update the spatial position information (13aₓ,13a_{y},13a_{z}) of the displaceable catheter in real time.

At step 8 the information on the procedure is being feed-back to the operator of the intervention. Preferably, the user-interface 30 comprises relevant clinical data, comprising the actual electrical activity of the tissue of the cardiac chamber 31,33,35 and positions of the detectable markers 5a,5b,5c,5d,7a,7b,7c,7d and a position of the displaceable catheter 13a. Preferably, the electrical activity is presented using a grey-coded representation, or using a suitable colour-code the corresponding ranges being given in R1, R2, R3 . . . RN windows. Also, the envisaged spatial roadmap 40a and the actual path of the displaceable catheter 40b are being presented. In case there is a mismatch between the path of the catheter 40b and the spatial roadmap 40a, the operator is signalled. After correcting for the mismatch, the interventional procedure is resumed.

FIG. 2 presents a schematic view of an embodiment of a system 100 according to the invention. For this particular embodiment an X-ray imager 100a is selected. As is indicated earlier, other medical imaging modalities, like magnetic resonance imager or an ultra-sonic machine are also suitable for practicing the invention. The X-ray imager 100a is arranged to form two-dimensional X-ray transmission images of a patient 130, which is positioned on the patient support table 114. The beam of X-rays 105 passes through the patient 130 and is intercepted by the X-ray detector 113. The X-ray detector 113, may be for example, a series arrangement of an X-ray image intensifier that feeds a television chain, while signals furthermore are A/D converted by means of an A/D converter 140 and are subsequently stored in suitable memory means 150. Conventionally, in order to produce a three-dimensional image of a target volume of the patient two orthogonal images of the patient are acquired. A movement of the X-ray source 112 around the patient 130 is enabled by the C-arm 101, which is rotatably mounted on a stand 111. Alternatively, in order to ensure higher reconstruction accuracy, a set of transmission images at different angulations is acquired. For this purpose the C-arm 101 is continuously rotated thus forming a rotational scan as is depicted by arrow 120, comprising a plurality of two-dimensional transmission images. In case the rotational scan is used for practicing the invention, the resulting images correspond to the series Dᵢ₋₁, Dᵢ, . . . , DN. These plural X-ray transmission images show the volume under examination, comprising the catheters 182a, 182b. These X-ray images are then processed by means of per se known reconstruction method to yield a motion-corrected three-dimensional volume of examination. This volume is then presented by means of suitable user-interface 181 on a display unit 183. Preferably, the user interface is arranged to provide a three-dimensional image of the target organ 184 together with distal portions of the catheters 182a, 182b provided with detectable markers 182a′, 182b′ (for simplicity only one detectable marker per catheter is shown). The motion-corrected three-dimensional image of the target organ 184 is used to construct the motion-corrected target organ-oriented three-dimensional coordinate system which is then used for drawing the spatial roadmap 183 and which is also used to locate a spatial position of a displaceable catheter (not shown), provided with a further detectable marker 185′. These computations are carried out using computing means 160. The operation of the imaging unit 100a is controlled by means of a control unit 117, which controls a movement of the C-arm 101 and the operation of the computing unit 160 arranged to carry out suitable data handling, including performing a three-dimensional reconstruction and motion compensation. The computing means 160 can be further arranged to carry out a further computation comprising a computation of a spatial discrepancy between the envisaged spatial roadmap 183 and the position of the displaceable catheter 185. This can be achieved by applying per se known rendering techniques. In case a substantial discrepancy is signalled and in case the catheters are positioned within the target organ by means of a controllable navigation system 190, the computing means calculates a control signal to be applied to the navigation system 190 to correct for the mismatch between the spatial roadmap 183 and the position of the displaceable catheter 185. Preferably, a stereotactic navigation system is used to control the positioning of the catheters within the target organ. The control unit then applies a correction signal S to the navigation system 190 after which an interventional procedure carries on. Preferably, the correction signal S is computed using an a-priori determined equation, alternatively a suitable look-up table (not shown) is addressed. It is also possible to guard the position of the catheters 182a, 182b in space. For this purpose the computing means 160 is arranged to perform a consistency check of the spatial position of the detectable markers of the catheters. In case a movement of a catheter is determined, the computing means reports this event to the control unit 117, after which a suitable control signal (not shown) is applied to the navigation system 190 to bring the moved catheter into its original position. Further details on the catheter control will be discussed with reference to FIG. 4.

FIG. 3 presents a schematic view of an embodiment of a user interface of a system according to the invention. The user-interface 200 is arranged to provide a real-time feedback of the course of the envisaged intervention to the operator. For this purpose the user-interface preferably comprises a read-out and controls screen 201 and a graphics screen 202. The graphics screen 202 can be arranged to present two-dimensional images of the organ 204 under investigation and/or three dimensional images of the organ 204. For simplicity of comprehension of the figure, a two-dimensional image is presented. The two-dimensional image comprises a suitable cross-section of the organ 204 together with catheters 206a, 206b used as reference catheters to construct a motion-corrected target organ oriented three-dimensional coordinate system, which is used for calculating and presenting the envisaged spatial roadmap 210. The catheters 206a, 206b comprise a plurality of detectable markers of a type 207a, 207b which are used as features to perform the motion correction. Also, a real-time spatial position of the displaceable catheter 208, for example as used for ablation during an electrophysiologic intervention, is given. The displaceable catheter 208 also comprises detectable markers 208a which are also projected on the graphics screen. In order to enable an easy following of the intervention, the read-out and controls screen comprises a plurality of dedicated fields 220, 222, 224. The first dedicated field 220 comprises a first plurality of sub-areas 220a-220f whereto useful information about the system is projected. Such information may comprise data on the position of the C-arm, controls of the catheter navigation system guarding the consistency of the spatial position of the reference catheters 206a, 206b, relevant patient data including readings of monitoring devices, like ECG, or any other useful information. The second dedicated field 222 comprises a second plurality of sub-areas 222a-222d whereto actual data on the intervention are projected. This actual data may comprise the results of the measurements of the electrical activity of the cardiac chamber for purposes of conducting electrophysiology. It may also comprise diagnostics delivered by the quality control system, presenting the information on the spatial accuracy of the system according to the invention. The operation of the quality control system will be discussed in further detail with reference to FIG. 4. In case the quality control system signals a substantial discrepancy between the spatial position of the displaceable catheter 212 and the envisaged spatial roadmap 210, it is signalled in one of the sub-areas 222a-222d. As a result, a correction value to be applied to the catheter navigation system is highlighted in the control field 224. The operator has a choice to apply the suggested correction, or to bypass it. This is enabled by a dialogue sub-area 224c of the control field 224. It is also possible that a displacement of one of the reference catheters 206a, 206b is reported during the intervention. The operator then addresses the quality control system to perform a recalibration of the motion-corrected target organ oriented three-dimensional system, which is enabled in any of the control fields 224a-224c. After the recalibration is performed, the spatial position of the spatial roadmap 210 is accordingly adjusted and the intervention carries on.

FIG. 4 presents a schematic view of an embodiment of a quality control system according to the invention. The quality control system 160′ according to the invention is integrated into the functional elements of the system 100, in particular of the computing means 160 and functions within it. The operation of the system 100 is described in detail with reference to FIG. 2. In this embodiment of the system 100, the computing means 160 comprise means for recording a spatial position of the detectable markers 162, which is arranged to analyze the individual coordinate of each of the detectable markers of the reference catheters 182a, 182b within the computed motion-corrected target organ oriented three-dimensional coordinate system. The quality control system 160 further comprises means 162′ for monitoring the spatial position of the detectable marker, which can be implemented as a separate unit or a separate soft-ware, or can be a part of the recording means 162. The quality control system 160′ according to the invention further comprises means 164 for signalling a displacement of any of the detectable markers 182a, 182b during the intervention. For this purpose the computation means 160 performs a consistency check, directed to recalculate the coordinate of each detectable marker for a new image acquisition. In case a displacement of the detectable marker is detected, the means 164 actuates means 166 for calibration of the motion-corrected organ-oriented three-dimensional coordinate system in order to yield a new motion-corrected organ-oriented three-dimensional coordinate system. This recalibration is carried out using the recorded spatial position of the not moved detectable markers. When the new motion-corrected organ-oriented three-dimensional coordinate system is established, means 168 perform a calibration of the spatial roadmap 183 for the new motion-corrected organ-oriented three-dimensional coordinate system. The new spatial roadmap 183 is then presented on the user interface 181. Preferably, the quality control system 160′ comprises means 170 for conforming a path of a displaceable catheter to the spatial roadmap. Means 170 can be arranged to provide a plurality of commands to the operator instructing him how to position the displaceable catheter. Preferably, means 170 is arranged to control the navigation system 190 thus automatically positioning the displaceable catheter in three-dimensions. In order to communicate with the quality control system according to the invention, the navigation system 190 is adapted with a control unit 192 arranged to manoeuvre the catheter in accordance with a received control signal from the quality control unit. It is also possible that means 170 supply a trigger signal (not shown) to the central unit 117, which in turn applies a corrective signal to the control unit 192 of the navigation system 190.

The present invention has been disclosed with reference to preferred embodiments thereof. Persons skilled in the art will recognise that numerous modifications and changes may be made thereto without exceeding the scope of the appended Claims. In consequence, the embodiments should be considered as being illustrative, and no restriction should be construed from those embodiments, other than as have been recited in the Claims.

1. A method for generating a spatial roadmap (12) representing an envisaged trajectory of an interventional device (13di) within a target organ (1), said method comprising the steps of:
acquiring image data (Dᵢ₋₁, Dᵢ, Dᵢ₊₁) of detectable markers (5a-5d, 7a-7d) arranged within the target organ (1);
constructing a motion-corrected target organ-oriented three-dimensional coordinate system (10) using said image data (Dᵢ₋₁Dᵢ, Dᵢ₊₁);
deriving a respective spatial position information (5cₓ,5c_{y},5c_{z}) of the detectable markers within the motion-corrected target organ-oriented three-dimensional coordinate system (10);
constructing the spatial roadmap (12) within the target organ (1) by interrelating the respective spatial position information (5cₓ,5c_{y},5c_{z}) of the detectable markers (5a-5d, 7a-7d).

2. A method according to claim 1, said method further comprising the steps of:
acquiring a set of readings (31,33,35) at their respective measurement locations within the target organ using an interventional measurement catheter;
presenting the set of readings on the spatial roadmap (40a).

3. A method according to claim 1, wherein the method further comprises the steps of:
acquiring further image data (I1,I2) of a displaceable catheter (13di) in the target organ (1) for a dwell position of the displaceable catheter, said displaceable catheter comprising further detectable markers (13a), said further image data comprising images of detectable markers (5a-5d, 7a-7d) and further detectable markers (13a);
deriving further respective spatial position information (13aₓ,13a_{y},13a_{z}) of the further detectable markers of the displaceable catheter within the motion-corrected target-organ oriented three-dimensional coordinate system (10).

4. A method according to claim 3, wherein the method further comprises the step of:
matching further respective spatial position information to the spatial roadmap automatically (40a,40b).

5. A method according to claim 1, wherein for purpose of derivation of a motion-corrected target organ-oriented three-dimensional coordinate system (10) an image acquisition by means of a rotational scan (I) of an X-ray source around the target organ is carried out.

6. A method according to claim 1, wherein for purpose of derivation of a motion-corrected target organ oriented three-dimensional coordinate system (10) an image acquisition of the target organ by means of a magnetic resonance apparatus is carried out.

7. A system (100) for generating a spatial roadmap representing an envisaged trajectory of an interventional device within a target organ, said system comprising:
a catheter (182a,182b, 185) arranged with detectable markers, said detectable markers being conceived to be positioned within the target organ;
a data acquisition system (100a, 113) arranged to acquire image data (Dᵢ₋₁, Dᵢ, Dᵢ₊₁, I, I1, I2) comprising the detectable markers;
computation means (160) arranged to:
construct a motion-corrected target organ-oriented three-dimensional coordinate system (10) based on said images;
derive a respective spatial position information (207aₓ, 207a_{y}, 207a_{z}, 207bₓ, 207b_{y}, 207b_{z}) of the detectable markers within the motion-corrected target organ-oriented three-dimensional coordinate system (10);
construct the spatial roadmap (210) within the target organ by means of interrelating the respective spatial position information of the detectable markers.

8. A system according to claim 7, wherein said catheter is further arranged to acquire readings at their respective locations within the target organ, said computation means (160) being further arranged to present said readings on said spatial roadmap.

9. A system according to claim 7, wherein the system further comprises a displaceable catheter (208) conceived to be displaceably arranged within the target organ (204), said displaceable catheter being arranged with further detectable markers (208a), the data acquisition means being further arranged to acquire further image data of the detectable markers and the further detectable markers for a dwell position of the displaceable catheter, the computation means being further arranged to derive further respective spatial position information (208aₓ, 208a_{y}, 208a_{z}) of the further detectable markers within the motion-corrected target organ-oriented three-dimensional coordinate system (10).

10. A system according to claim 7, wherein the computation means (160) is further arranged to match the further respective spatial position information of the further detectable markers to the spatial roadmap (210,212).

11. A system according to claim 7, wherein the system further comprises navigation means (190) conceived to position the catheter and/or the displaceable catheter (182a,182b,185) within the target organ.

12. A system according to claim 11, wherein the computation means is arranged to control (S) the navigation means in order to conform the further spatial position information to the spatial roadmap (210,212).

13. A system according to claim 7, wherein said system further comprises a user interface (30,200) arranged to feedback a three-dimensional image of the spatial roadmap (40a, 210) and the spatial position of the catheter and/or the displaceable catheter.

14. A system according to claim 13, wherein the user interface is arranged to present a further three-dimensional image comprising the target organ (204).

15. A quality control system (160′) arranged to guard a spatial accuracy of a system as claimed in claim 7, said quality control system comprising:
means (162) for recording a spatial position of detectable markers;
means (162′) for monitoring the spatial position of the detectable markers;
means (164) for signalling a displacement of any of the detectable markers during an intervention;
means (166) for calibration of the motion-corrected organ-oriented three-dimensional coordinate system to yield a new motion-corrected organ-oriented three-dimensional coordinate system using the recorded spatial position of the detectable markers;
means (168) for calibration of the spatial roadmap for the new motion-corrected organ-oriented three-dimensional coordinate system.

16. A quality control system according to claim 15, wherein said system further comprises means (170) for conforming a path of a displaceable catheter to the spatial roadmap.

17. A quality control system according to claim 16, wherein the displaceable catheter is being positioned by means of a guiding system (190), the means (170) for conforming a path of the displaceable catheter to the spatial roadmap being arranged to communicate (S) to said guiding system (190).
